(19) United States
(12) Patent Application Publication
(10) Pub. No.: US 2023/0346604 A1
(43) Pub. Date: Nov. 2, 2023

(54) METHODS AND APPARATUS FOR ANTI-FOGGING EYEWEAR

(71) Applicant: Jeffrey Michael Simpson, Sarasota, FL (US)

(72) Inventor: Jeffrey Michael Simpson, Sarasota, FL (US)

(21) Appl. No.: 18/141,902

(22) Filed: May 1, 2023

Related U.S. Application Data
(60) Provisional application No. 63/337,580, filed on May 2, 2022.

(51) Int. Cl. A61F 9/02

(52) U.S. Cl. CPC A61F 9/028

(57) ABSTRACT

Anti-fogging apparatus and methods for protective eyewear which use air flow directed by designed nozzles to the surface of the lens of the protective eyewear are described. The established flow into the mask reduces or eliminates fogging on a lens of the mask. In some examples, air is directed into the interior protective area using nozzles that are positioned at particular angles relative to the lens. Said apparatus operates under particular configurations and with described methodology to improve anti-fogging ability while creating minimal disruption to the user's eyes.

### CROSS-REFERENCE TO RELATED APPLICATIONS

This application claims the benefit of U.S. Provisional Patent Application No. 63/337,580 entitled Anti-Fogging Safety Eyewear Insert and filed on May 2, 2022. The contents of which are incorporated herein by reference.

### FIELD OF THE DISCLOSURE

The present disclosure relates generally to apparatus associated with facial coverings to mitigate or prevent fogging and methods for their use. More specifically, the present disclosure is directed to a nozzle system to allow air to be directed across a lens of protective facewear at particular velocities and angles to prevent or minimize fogging on the lens.

### BACKGROUND OF THE DISCLOSURE

Commercially available protective eyewear presents users with a difficult choice: unsealed or fully. Unsealed protective eyewear provides ventilation for the protected area, at the expense of admitting small particles into the protected area. As a result, unsealed protective eyewear may be unhelpful in environments containing hazardous particles. Such environments include, for example, underwater environments, buildings that are on fire, or combat zones (including simulated combat zones, such as Airsoft games). These environments contain particles that may irritate or damage the wearer's eyes.

In such environments, it is generally preferable to use fully sealed protective headwear. Fully sealed protective eyewear attempts to prohibit ingress of most materials into the protected space. On the other hand, this may mean that moisture can build up easily inside the protected space and cannot easily escape. This can lead to fogging of the lenses of the protective eyewear, which can effectively blind or substantially decrease the wearer's field of vision.

To address this fogging problem, some have tried including ventilation holes to alleviate fogging issues by providing a source of external air to keep moisture from building up in the space between the wearer's face and an inner surface of the lens of the protective eyewear. These systems are generally insufficient to prevent fogging.

Others have tried using external air flow systems, which roughly speaking connect an external fan to the protected area with a tube. These systems are bulky, cumbersome, and fail to adequately provide an acceptable interface between the external air source and the interior region of the protective eyewear. This bulk can make such systems unworkable in certain environments, such as fire-fighting and military applications.

### SUMMARY OF THE INVENTION

Accordingly, what is needed is a secure, convenient, and safe assembly to attach an air flow tube from an external fan and direct that air flow appropriately onto the lens.

Protective eyewear may be found in a variety of types. In some examples, closed environment protective eyewear may include masks of various types, goggles, pairs of goggles and the like. Some closed protective eyewear may draw air from an environment of the user while other examples may have gas sources to provide the air for the system, such as bottled air or oxygen. The various types of contained environment eyewear may form condensates of water vapor on the surfaces of lens. The condition may also be referred to as fogging of the eyewear. The present disclosure provides apparatus and methods to efficiently defog lens surfaces and to keep them defogged for extended periods of time. Defogging according to many examples of the present disclosure is accomplished by directing a flow of air through a nozzle in a configured orientation with respect to the lens surface to be defogged. The flow of air may be further configured by controlling the rate of air flow flowing through the nozzle. The established air flow in the direction of the surface of the lens creates a specific flow condition which follows the lens surface and lowers the pressure in the vicinity of the lens surface and the result is a defogging the lens surface where the directed air flow occurs. A major portion or all of the optic zone of the lens, being the portion of the lens through which the user observes images, may be defogged in this manner with appropriate design and configuration of nozzles. Accordingly, in some examples, the lens surface may be kept free of condensation for extended periods of time by the maintenance of the air flow conditions. There may be numerous other systems that are configured with the air flow system including regulators, fans, sensors, dryers, filters, and the like.

In some examples, similar devices with air flow inserts which include nozzles and air flow receivers to connect to tubing or other conducts and the other configured system components may be formed for open environment type eyewear.

One general aspect includes a method of controlling a fogging condition on a lens surface. The method also includes flowing air out of a nozzle across the lens surface, where the nozzle directs the air towards the lens surface and reduces a pressure at a region of an interface between the lens surface and the flowing air; and maintaining the flowing of air such that a condensate of water vapor on the lens surface is evaporated into the flowing air.

The method may include viewing with a user's eye an image through an optic zone of the lens surface, where a clarity of the view of the image is improved by the evaporation of the condensate. The lens surface may be included within a closed environment of a mask. In some examples, a rate of flowing air is less than an amount equal to a volume of the closed environment per minute. In other examples the rate of flowing air may be an amount or range of amounts between flowing an amount equal to the volume of air in a range between 10 seconds and 20 minutes. Some typical flow rates may include roughly 1.3 liters per minute for a common face shield. In some examples, a flow rate may be specified for a particular mask as a target value or a target range of values that may be within 0.1 liters per minute and 100 liters per minute.

The lens surface may be included within an open environment.

In the various examples the rate of flowing air flows across the surface at a rate of roughly 1.4 m/sec. In other examples, the targeted rate of flow across the surface may be an amount or a range of amounts between 1 mm/sec and 10 m/sec.

The nozzle may be included within an air flow insert, where the air flow insert further may include an air flow receiver where air is provided to flow to the nozzle, and where the air flow insert is affixed to the lens surface.

The nozzle may be included within an air flow insert, where the air flow insert further may include an air flow receiver where air is provided to flow to the nozzle. In some examples, the air flow insert may be included within a mask. In some examples, the air flow insert passes through a bulkhead of the mask.

In some examples, the air flow insert may be included within a goggle where the air flow insert passes through a bulkhead of the goggle.

One general aspect includes a method of producing an anti-fogging apparatus. The method may include configuring a structure where the structure may include at least a lens element. The method may further include configuring an air flow insert, where the air flow insert may include at least an element to attach to the lens element, a nozzle, and an air flow receiver. The method may also include attaching the air flow insert to the structure. In some examples the method also includes examples where after the attaching, the nozzle is positioned to guide air flow through the nozzle towards a surface of the lens element and across a span of an optic zone of the lens element.

Implementations may include one or more of the following features. The method may include attaching a regulator to the structure, where the regulator controls a rate of the air flow through the nozzle. In some examples, a user control of the regulator may adjust the rate of air flow through the nozzle. The adjusted rate of air flow through the nozzle may project air flow in a region of surface of the lens element. In some examples, the projecting air flow reduces a pressure of air in a region proximate to the lens surface. A fogging of the lens surface is removed upon flowing of the air flow. The flowing of the air flow on the lens surface may also prevent a fogging of the lens surface during a use of the anti-fogging apparatus for at least an hour of usage. In some examples, the prevention of fogging of a lens may be specified as a range of values between 1 minute and 6 hours or at any particular value between these ranges. In some examples, the prevention of fogging may occur for the entire time that a structure is worn and employed.

One general aspect includes an eye protection apparatus with a lens surface anti-fogging means. The eye protection apparatus also includes an eye protection housing. In some examples a lens may be configured on a first portion of the eye protection housing,

One general aspect includes an eye protection apparatus with a lens surface anti-fogging means. The eye protection apparatus also includes an eye protection housing; a lens configured on a first portion of the eye protection housing; and a nozzle positioned proximate to the lens. In some examples, the nozzle may include a receiving portion and an outflow portion, where the outflow portion is positioned at a flow angle relative to the lens surface. The example may further include an air flow regulator which may include an air driver; and an air supply line in fluid connection between the nozzle and the air flow regulator. The example may further include examples where the air supply line receives a flow of air driven by the air driver and connects the flow of air to the receiving portion of the nozzle. In some examples, the flow of air proceeds through the nozzle and projects upon the lens surface. The method may further include examples where the air flow reduces a pressure of air in a region proximate to the lens surface. In some of these examples, implementations may include examples where the reduction of the pressure of the air in the region proximate to the lens surface facilitates an evaporation of a condensate upon the lens surface and a reduction of a fogging. The method may also include examples where the reduction of the pressure of the air in the region proximate to the lens surface facilitates an evaporation of a condensate upon the lens surface and a reduction of a fogging. The method may also include examples where the reduction of the pressure of the air in the region proximate to the lens surface inhibits a condensation of a condensate upon the lens surface and an inhibition of a fogging. The method may include examples that may include a strap, where the strap holds the eye protection apparatus into a position configured upon a head of a user such that the user may view an image through an optic zone of the lens surface, where the optic zone of the optic zone of the lens surface is free of the fogging. The method may also include examples where the lens surface is may include within a closed environment or where the lens surface may be included within an open environment. The method may also include examples where the reduction of pressure of the air in the region proximate to the lens surface is more than 5 torr above that necessary to prevent condensation, and where the reduction of pressure of the air occurs across all of the optic zone of the lens surface. In still further examples the reduction of pressure of the air in the region proximate to the lens surface may be more than 0.1 torr or may be a range of values between 0.1 torr to 100 torr.

### BRIEF DESCRIPTION OF THE DRAWINGS

The accompanying drawings, which are incorporated in and constitute a part of this specification, illustrate several embodiments of the invention and, together with the description, serve to explain the principles of the invention:

FIG. 1A—An illustration of an exemplary mask with defogging air flow inserts.

FIG. 1B—An illustration of an exemplary regulator.

FIG. 1C—An illustration of an exemplary nozzle.

FIGS. 2A-2C—Illustrations of an exemplary mask according to aspects of the present disclosure.

FIGS. 3A-3E—Illustrations of an exemplary goggle according to aspects of the present disclosure.

FIG. 4—An illustration of an exemplary self-contained breathing apparatus according to aspects of the present disclosure.

FIGS. 5-7—Illustrations of an exemplary mask according to aspects of the present disclosure.

FIG. 8—An illustration of an exemplary protective eyewear with direct attachment of an air receiver.

FIG. 9—A closeup of a nozzle within an exemplary mask.

FIG. 10—An illustration of an exemplary protective eyewear including a prescription optic component.

FIGS. 11A-11B—An illustration of an exemplary eyewear including optic components according to aspects of the present disclosure.

FIG. 12—An illustration of exemplary processing apparatus according to aspects of the present disclosure.

FIGS. 13-20—Flow diagrams of exemplary methods according to aspects of the present disclosure.

### DETAILED DESCRIPTION

The present disclosure relates to methods and apparatus for providing an anti-fogging eyewear insert.

In the following sections, detailed descriptions of examples and methods will be given. The description of both preferred and alternative examples, though thorough, are exemplary only. It may be understood that, to those skilled in the art, variations, modifications, and alternations may be apparent. The examples given do not limit the broadness of the aspects of the underlying invention, as defined by the claims.

Throughout this disclosure, references may be made to certain types of protective eyewear. These references are not meant to be restricting. Ultimately, protective eyewear refers to any covering that shields the eyes of a wearer from an exterior environment including underwater environments or a vacuum. The shielded area is referred to herein as the protective area. Protective eyewear may differ based on various characteristics, such as characteristics of the user (e.g., face size), characteristics of a deployment environment (e.g., paintball game, underwater, or blazing fire), or comfort. Protective eyewear can also differ on the amount of protection actually conferred; for example, some eyewear shields primarily the eyes (e.g., Olympic swimmer goggles), while other eyewear shields more of the user (e.g., a firefighter helmet and face shield). While the present disclosure addresses some of these specific types of eyewear as examples, it can readily be understood that the general scope of the present disclosure can apply to a wide variety of types of eyewear.

The present disclosure may operate in open or closed loop systems. In general, an open system utilizes ambient local atmosphere, at a current ambient air pressure, and applies air to a lens of a protective mask. This air may then be ventilated back into the ambient local atmosphere. In some embodiments, this may involve the use of a fan (impeller, axial, or turbine) or other air movement apparatus to create an air flow.

In general, a closed system utilizes a pressurized air source (e.g., a compressed air cartridge or a compressed air tank). A closed system may use a fan, or it may use the existing pressure within the closed system to recirculate air by creating pressure gradients within the system. Closed systems may recirculate air through a regulator in a low-pressure system. Such air recirculation may utilize an air flow return line back to a regulator which, in a low-pressure or atmospheric pressure system, may be implemented using an impeller fan. From time to time, closed systems with air recirculation may purge the air supply from the system and reintroduce air into the system from an external air supply, such as a pressurized air tank or cartridge. This may occur based upon a lapse of time, a change in ambient atmospheric pressure (as measured by a sensor), manual control by a user, or other similar metric.

Unless otherwise specifically described herein, nozzles may generally be attached to a frame or a lens component of protective headwear integrally (i.e., built in), through mechanical means, through adhesive means, or otherwise. Air flowing through the nozzles may do so, for example, by tubes or other lining that enter a protective area of the mask to connect with the nozzles by means of a gland, such as an articulating gland.

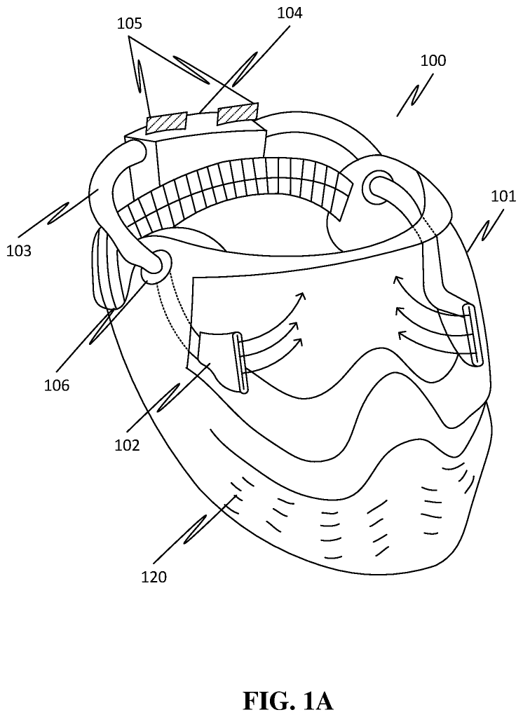

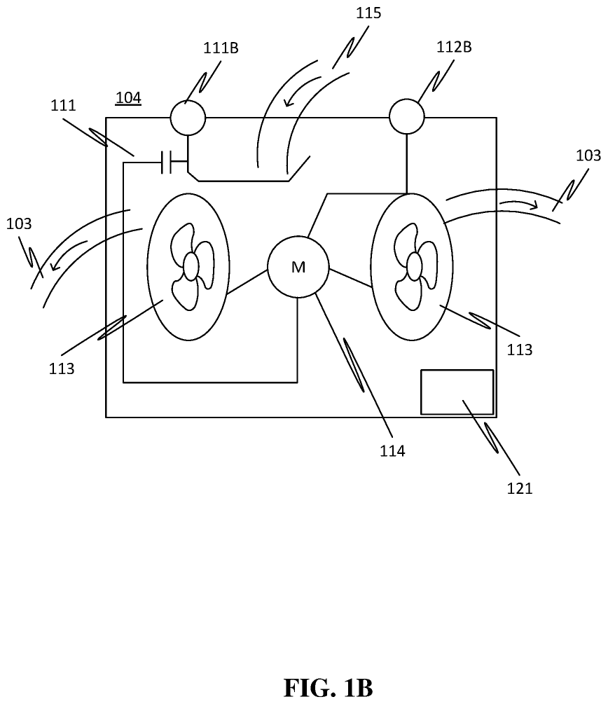

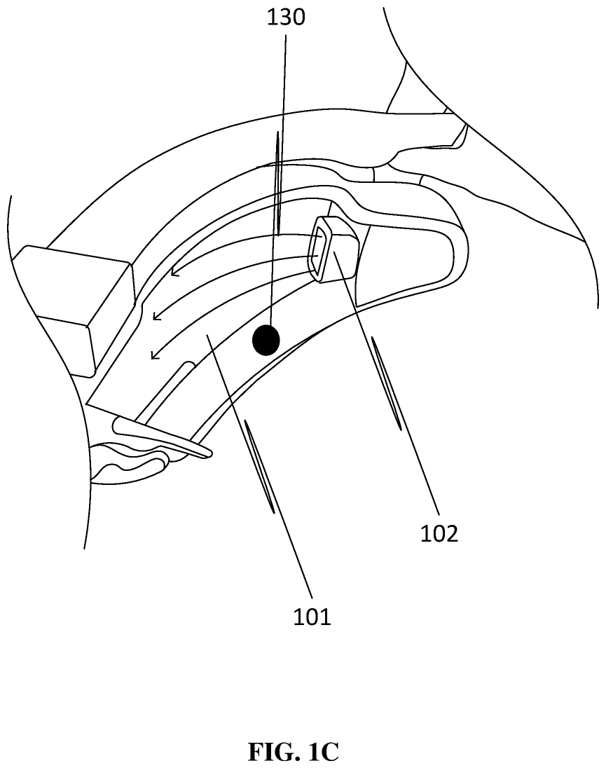

Referring now to FIG. 1A, an exemplary embodiment of the present disclosure is shown. While this particular embodiment depicts a mask suitable for deployment in, e.g., a paintball game, it may be understood that the mask may be suitable for deployments in other situations having similar needs. Mask 100 may include lens 101, which creates a protective area between lens 101 and a face of the wearer. Within the protective area, one or more nozzles 102 may shoot air across a surface of lens 101. Nozzles 102 may be fed by one or more air supply lines 103, which fluidly connect nozzles 102 with an air flow regulator 104. The mask may also have control features such as control buttons 105. In some examples, the supply lines may pass through features of the mask and be sealed with the use of a gland 106.

Referring briefly to FIG. 1B, a simplified diagram view of an exemplary regulator 104 is shown. Regulator 104 may include a substantially sealed compartment for driving one or more air movement devices, such as fans 113, which in turn drive one or more air circuits through an attached mask such as mask 100. Fans 113 may represent any apparatus suited for moving air or creating pressure gradients, such as turbine fans, impellers, and piezoelectric fans. The fans 113 may be connected to power source 111, such as a battery, and may be driven by motor 114. Power source 111 and motor 114 may be controlled by control systems 111B and 112B, respectively. Control systems 111B and 112B are depicted in FIG. 1B as buttons; however, they may also be switches or dials or other features that can be used to alter a control condition. Exemplary control systems 111B and 112B may also be controlled by a wireless communication means, such as Bluetooth, as described in more detail below. Regulator functionality, including communications aspects, may be controlled by a micro-controller unit 121. Alternative air movement devices may be used in conjunction with fans 113. In some examples instead of fans 113 the air movement devices may include compressed air.

Control system 111B may be a simple on/off toggle; i.e., which may be used to actuate control system 111B. When the control system is actuated, power source 111 may begin driving the components shown in FIG. 1B. Control system 112B may, in some embodiments, provide a finger control for driving motor 114. For example, in some embodiments, control system 112B may be actuated in stages to affect a quality of the air being driven by motor 114 via fans 113, such as by affecting a speed of the fan, an implementation of an air filter, or other similar quality changes. Motor 114 may be any device capable of driving fans, such as, for example, an EMF motor.

In some embodiments, control system 112B may be further operated via one or more sensors located within regulator 104 or elsewhere on mask 100. For example, mask 100 may include temperature or humidity sensors to monitor an amount of air, moisture, or heat within the protective area. In some examples, the sensors may be connected to the control system 112B and provide feedback signals that may be used by the control system to adjust condition of the air flow. In some other examples, the sensors may wireless transmit data related to their sensing to a wireless receiver located within regulator 104. After receipt of the data, the regulator may process the received data and determine adjustments to make an amount, velocity, or pressure of air passed through mask 100. In some other examples, the sensors may communicate with other devices such as a smart device, computer, or other device capable of wireless connection to the sensors. For example, if these sensors detect an increased amount of humidity (i.e., moisture) within mask 100, then the sensor may transmit a command (or a wireless receiver located within regulator 104 may transmit a command, based upon receipt of the sensor data) to control system 111B to increase the speed of motor 114.

In some embodiments, a processor in logical connection with the wireless receiver or the sensor, together with a wireless communication means and storage containing software, may adjust the fan 113 or motor 114 speed based upon a desired optimization of power source 111. For example, if power source 111 is a fully charged battery, then detection of increased humidity by a sensor, may trigger a throttling of the fan sufficiently to apply air to lens 101 to eliminate all moisture fully or nearly fully on lens 101. In contrast, if power source 111 is a battery with 20% of its charge remaining, then detection of increased humidity by a sensor may result in a less-than-100% elimination of humidity to preserve the remaining life of power source 111.

Regulator 104 may be in connection with one or more air supply lines 103. Air supply lines 103 may be tubes made out of a suitable material, such as polyvinyl chloride (PVC), polyurethane, nylon, or polyethylene. In some embodiments, it may be desirable for air supply lines 103 to be flexible; for example, in dynamic environments such as a paintball game, users may be required to move quickly and flexibly. Such users may not be able to wear a mask that restricts their movements. Accordingly, more flexible air supply lines 103 may be desirable in such deployments. In some examples, air channels may be molded into the protective mask and/or lens.

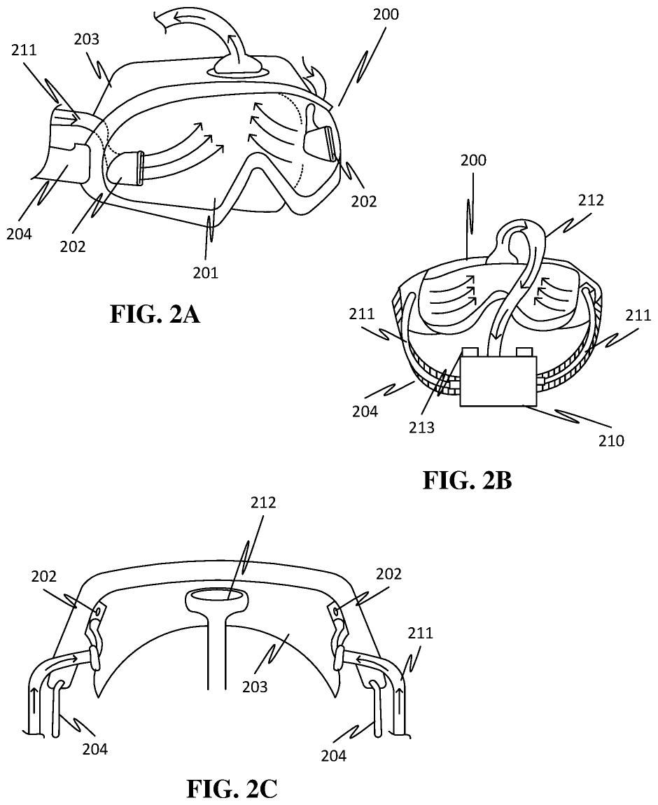

In some embodiments, regulator 104 may optionally include return nozzle 115. Like air supply lines 103, return flow tube 115 may also be made a flexible tubing material, such as PVC, polyurethane, nylon, or polyethylene. In such embodiments, air flows from the protective area in mask 100 back into regulator 104 as a way of venting the protective area and mitigating over-pressurization risks. Such an embodiment is shown in, for example, FIG. 2.

Referring back to FIG. 1A, air that has been driven by fans 113 through air supply lines 103 may enter mask 100 via a gland (e.g., an articulating gland) or other air flow connector to allow the air to reach the interior of mask 100. Air may escape mask 100 in several ways to avoid over-pressurizing the protective area. In some embodiments, the air may flow through one or more ventilation holes 120. In embodiments discussed in more detail below, the air may also flow through a return flow tube, back into the regulator 104. In some embodiments, ventilation holes 120 may include one or more filters to prevent certain particles from entering the protective area. In some embodiments, ventilation holes 120 may include one-way air valves, or other similar structures to prevent harmful environmental aspects from entering the protective area.

Air may flow through nozzles 102 into the protective area. Nozzles 102 may be attached to the mask frame or directly to lens 101 via mechanical means, adhesive, or otherwise. Nozzles 102 may also be built directly into the mask frame. A close-up view of an exemplary nozzle 102 is shown in FIG. 1C. An opening of nozzle 102 proximate to lens 101 may be shaped as shown in FIG. 1C to provide designed volumes or velocities of air to interact with the region near to or on lens 101. Moreover, an angle of nozzle 102 (or an angle of the proximate opening) relative to the lens may be chosen in connection with such volumes or velocities to minimize amounts of moisture or other condensation occurring on the lens. For example, in the embodiment shown in FIG. 1C, nozzle 102 is configured to control air flow characteristics including for example a relative angle of the air flow as it approaches the surface of the lens 101. In some examples, the rate of the air flow through the nozzle 102 may be critical for obtaining an optimal defogging condition or to prevent condensation in the first place. There may be adjustments that are made during a production of a device to adjust the air flow for optimal defogging. In other examples, the air flow may be adjustable with controls on the headset device. In some examples, the rate of flow may be adjusted through the nozzle to be at least a minimum required for functional defogging but also is no more than required to optimize the comfort aspects of the air flow to the user, such as drying of the eye of the user. The shape and geometry of features of the nozzle may change for several reasons and applications. For example, if a greater distance of air flow across a lens is required, the nozzle may be designed as a smaller opening to reduce the cross-section area of the nozzle opening. Due to physical principles such changes may increase the effective velocity of the air flow. In some examples, an alternative degree of freedom may be a cross-sectional area of the nozzle opening which can be shaped to create a large or small fan (i.e., spread) of air flow across a lens to accommodate the required condensation free surface area on the lens to maintain the user's visual acuity.

The design and the geometry of the nozzle generates air flow that is determined by a combination of physical effects of air flow. In some examples, an effect of a nozzle may be to create an air flow that closely follows across the surface of the lens with a sufficient velocity as to decrease the effective air pressure at the air-lens interface. Physical principles of air flows tend to keep an established air flow close to the lens surface when it is directed along said surface. Furthermore, physical principles mean flow conditions proximate and across a surface will result in decreasing an effective pressure at the air/lens interface. In some examples, an important aspect of nozzle design is to create the conditions for these physical principles. The resulting effect of optimally configuring air flow with nozzle design and operating conditions may be that the air pressure may be decreased enough along the surface of the lens to result in removal of condensation or to result in the prevention of condensation forming in the first place even as the temperature and humidity level between the user's face and the lens increases.

It may be noted that in some examples, the nozzle design and operating conditions may create an operating state which is fundamentally different than simply flooding the space between a user's face and the lens. Further, it may be noted that the operating principles according to the present disclosure may create other advantageous aspects including the application of lower amounts of air flow. Elevated levels of air flow into the space between the user's face and the lens may cause the user's eyes to dry out. Furthermore, the directed aspect of some examples of the present disclosure are in contrast to air flow which is not directed in a controlled manner. Non-directed flow may cause evaporation from all surfaces, including a user's eye surface, and rapidly increase the available moisture content in the air in the space between the user's face and the lens which may actually make fogging aspects worse.

Mask 100 may optionally include sensor 130. As has been described, sensor 130 may include, for example, a temperature or humidity sensor to gauge a temperature or humidity level within the protective area. Sensor 130 may transmit this information to a receiver located within or around the regulator 104. In some examples, a response to a sensed condition within the sensor 130 may include an adjustment to a characteristic of air being blown through nozzle 102. In some embodiments, sensor 130 may include an accelerometer or other sensor for measuring acute applications of force. If such a sensor makes a measurement indicative of an injury or head trauma for a user of mask 100, then sensor 130 may wirelessly transmit an emergency signal to a third party, such as an emergency operator or game supervisor.

In some examples, one or more sensors, such as sensor 130, may be a functional combination of a number of devices capable of sensing different conditions within the airspace. In an example, an IMU (Inertial Measurement Unit) may be included which may sense movement of the apparatus. In some examples, the IMU sensor may be used for battery management of the regulator. For example, if a regulator is motionless, the state may not require moving air flow. When the user starts moving the air flow system, the IMU may also detect motion and automatically begin any necessary air flow. In some examples, the IMU may also detect movement that will potentially require more air flow and thus be an input into the air flow velocity control system.

The IMU can also detect impacts and other movements that may indicate potential damage (e.g., a traumatic head/brain injury) or some other potentially damaging violent movement. This detection can trigger several other actions including sending a trauma event via the wireless networking capabilities, signal an audible signal to nearby teammates or other personnel and/or record the event for analysis at some later time or place.

In other examples, sensing may include Humidity/Temperature sensing. The system may contain a plurality of these sensors to best ascertain whether the user and their protective equipment are at the condition where condensation may occur on the lens. Temperature and Humidity sensing may allow the regulator unit to adjust the air flow through the nozzle proximate to the lens to provide the most efficient air flow to maximize the efficiency and effectiveness of the system. The humidity and temperature sensors may allow for some embodiments to engage air conditioning devices that would heat/cool the air and/or humidify/dehumidify the air to provide the best air quality for the user.

In other examples, sensing may include gas and chemical sensors. In these examples, the gas and chemical sensors may alert the user to potential dangerous or harmful gases. These sensors may be used to switch a particular embodiment from an open to a closed system where the air flow source switches from the ambient atmosphere to a tank of compressed air to protect the user from potential harm from airborne chemicals. In similar examples, the sensing may include radiation sensing such as for alpha ray, beta ray or gamma ray radiation emissions. Other types of sensing may be used to sense aspects of the environment surrounding the various apparatus.

In some examples, the data flow from the one or more sensors located within the environments described in this disclosure may be processed utilizing one or more of algorithmic and or analytic techniques. For example, the data stream may be input to AI and/or Machine Learning algorithms to enhance the operation of the anti-fogging apparatus.

In some examples, the nozzle illustrated in FIG. 1C may depict how a nozzle shape may determine the shape of the air flow across the lenses. A shape and location of the nozzle may change for several reasons and for different applications. For example, if a greater distance across the lens is required, the nozzle may become more of a slit and have a smaller opening. In some examples this may reduce the cross-section area of the nozzle opening. The physical effect of reducing a cross sectional area of the nozzle opening may be to increase the effective velocity of the air flow. Also, that cross-section area of the nozzle opening can be shaped to create a large or small fan of air flow across a lens to accommodate the required condensation free surface area on the lens to maintain the user's visual acuity. Slots or holes may be used to independently direct air flow across a lens surface. In some examples, when the nozzle opening directs air flow into the vicinity of a lens, a physical effect may be to have a tendency of the air flow to stay localized across a lens surface even when the lens surface may be curved. It may be particularly effective to structure and aim a nozzle such that the air flow does stay in connection with the lens surface, effectively keeping the thermal and humidity conditions of the internal surface of the lens in a relatively effective state to limit fogging or hazing of the vision through the lens. In some examples, the nozzle characteristics may be adjustable such as by way of non-limiting example through electrical control of actuators. In similar manners the flow rate of air through the nozzle may be adjusted at the regulator to alter the flow characteristics in the region of the lens.

In some examples, the air may be further processed by elements that may be located in the regulator or attached in other locations along the frame of the eyewear. In a non-limiting example, a heating or cooling element in fluid contact with the air flow may adjust a temperature and/or change a humidity of the air. In a similar manner, a desiccant which may be located in a replaceable cartridge may be included in the air flow to reduce humidity, in particular for examples where the air flow is recirculated. Similar processing of the air flow may also be used to improve a functionality of the air flow directed by the nozzle, but a fundamental aspect of improvement is based on establishing nozzle design and operations to control the air flow to operate in the flow regime that maintains good interaction of the air flow with lens surfaces.

Referring now to FIG. 2A, an alternative embodiment of the present disclosure is shown. In the embodiment shown in FIG. 1, air may flow out of the sealed protective region via ventilation holes in the mask. However, some applications may require a more substantial seal between the protective region and an exterior, such that ventilation holes may not be appropriate. For example, in underwater environments, it may not be desirable to have ventilation holes in goggles.

Accordingly, the embodiment shown in FIG. 2A depicts goggle apparatus 200, which may be suitable for underwater deployment, especially in scuba environments. Goggle apparatus 200 may include lens 201, one or more nozzles 202, mask gasket 203, and a strap to hold the apparatus in place. Lens 201 may be made of transparent (or substantially transparent) polycarbonate or other materials suitable for deployment in a pressurized, underwater environment. Nozzles 202 may be shaped substantially similarly to the nozzles 102 described in the embodiment shown in FIG. 1C. Nozzles 202 may be fluidly connected to one or more tubes 211 to allow a gas to be transported from outside the protective area (i.e., the area between lens 201 and a face of the user) to within the protective area. In exemplary embodiments, the gas may be oxygen or atmospheric air; however, other gasses may be substituted if desired.

Strap 204 may attach to one or more positions on goggle apparatus 200. The strap 204 may allow for removable (and, in some embodiments, flexible) attachment of goggle apparatus 200 to a face of the user. Strap 204 may be adjustable in length to allow for fitting on a variety of face types.

Mask gasket 203 may sit in a position between lens 201 and a face of the user. Mask gasket 203 may also be fluidly connected to tubes 211 or nozzle 202. In some examples, the mask gasket may be formed of flexible materials capable of forming a seal with underlying skin and other surfaces of a user, such as silicone or thermo-plastic elastomers as non-limiting examples.

Referring now to FIG. 2B, in a rear view of the example from FIG. 2A, Goggle apparatus 200 may further include regulator 210, which may connect to one or more outflow tubes 211 and return flow tube 212. Optionally, regulator 210 may further include control system 213. Both outflow tubes 211 and return flow tube 212 may connect to goggle apparatus 200 (and to the protective area) via apparatus capable of facilitating substantially full seal on the system, such as via a gland.

Control system 213 may allow for the adjustment of air flow rates. Such adjustment may include the use of buttons or control knobs. In some embodiments, control system 213 may include a wireless or a wired communication means, such as a Bluetooth receiver or transceiver, which may allow for adjustments to control system 213 (and hence to regulator 210) to be made by a device capable of transmitting the corresponding wireless communication modality. For example, in some embodiments, a Bluetooth-equipped smart phone may be used to increase or decrease the rate of air flow applied to lens 201.

In this way, anti-fog capability may be achieved by creating a circulating air flow through goggle apparatus 200 from regulator 210. In exemplary embodiments, regulator 210 may be attached to strap 204, to an oxygen tank of the user, or any other similar place to allow air transport throughout the closed system described herein. In low-pressure configurations (such as shallow dives), no new air from a highly pressurized air source is required. Thus, air flow may be generated through a fan (which may be located within regulator 210 and fluidly connected to outflow tubes 211). In some embodiments, a speed of the fan may be controlled by control system 213. In some embodiments, more than one fan may be required (e.g., to move air through more than one outflow tube 211).

Air may flow from a fan in regulator 210, through outflow tubes 211, and into the protective area via nozzle 202. In exemplary embodiments, nozzle 202 may be positioned at an angle to allow for substantially all of the air flowing through nozzle 202 to be directed at lens 201. In this way, air flows across the lens with enough velocity and in such a manner to prevent condensation from occurring. Air may then flow back through return nozzle 212 to prevent excess pressure from building up in the protective area.

In some embodiments, goggle apparatus 200 may additionally include sensors, such as temperature or humidity sensors. These sensors may transmit data to regulator 210 to adjust a speed of air fed through nozzle 202, as described analogously in FIG. 1B.

Referring now to FIG. 2C, a top view of the goggle apparatus 200 is illustrated. In some examples, the outflow tubes 211 may be routed above the strap 204 and passed through the google lens with glands and to nozzles 202. The return nozzle 212 may also utilize a gland to pass through the mask gasket 203.

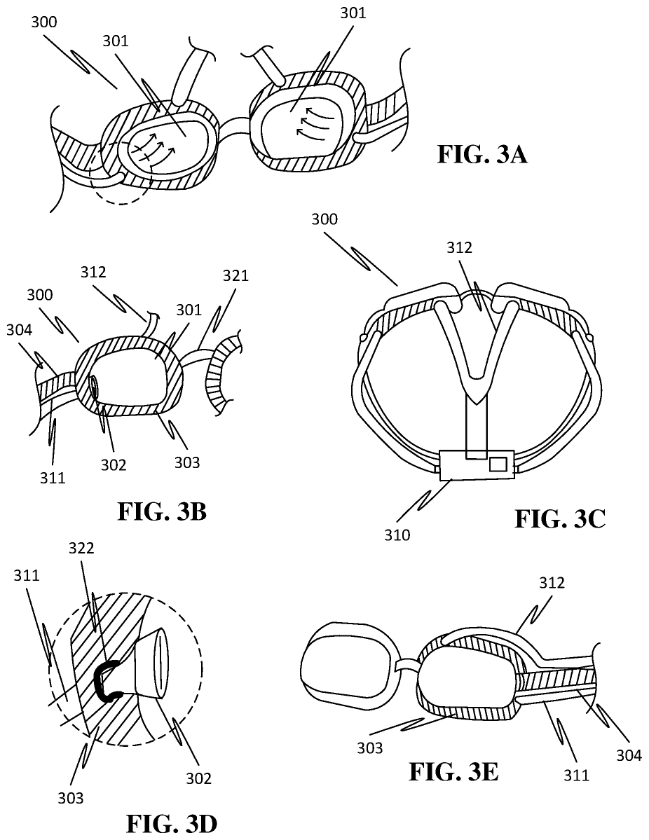

Referring now to FIG. 3A, an alternative embodiment of the present disclosure is shown. Like the embodiment shown in FIG. 2A, this embodiment adapts an air transport system similar to that shown in FIGS. 1A-1B. However, this embodiment is specially adapted for swimmer's goggles 300. Accordingly, each lens 301 may have its own air distribution system. In some embodiments, each lens 301 may also have its own regulator; however, in exemplary embodiments, one regulator may suffice to serve both lenses 301.

Referring now to FIG. 3B, a reverse view of a lens region of a swimmer's goggles 300 is illustrated. In each lens, mask gasket 303 may cushion the goggles 300 against a face of the wearer when held by strap 304. Mask gasket 303 may include nozzle 302 and return flow tube 312. Nozzle 302 may be fluidly connected to an outflow tube 311. In some examples, goggles 300 may generally include nose bridge connector 321. In some embodiments, nose bridge connector 321 may also be in fluid connection with the nozzles 302, such that air can flow freely between lenses 301. This may be useful in, for example, high water pressure environments.

Referring now to FIG. 3C, a top view of swimmer's goggles 300, air that is driven into the protective area bounded by lens 301 may return to regulator 310 via return flow tube 312. In an example, the return flow tube 312 (a combination of two parts, one from each goggle) may be long enough to loop over the head of the user.

Referring now to FIG. 3D, a close up of the nozzle 302 region of a goggle is illustrated. The outflow tube 311 which provides air flow to the nozzle may pass through the mask gasket 303. In some example a gland or gland seal 322 may allow the tubing or air flow structure to pass through the mask gasket 303 in a leak proof manner.

In some embodiments, for example FIG. 3E, mask gasket 303, outflow tube 311, or return flow tube 312 (or any combination thereof) may be run on or through strap 304. Return flow tube 312 may pass through a gland or glands or other mechanism to escape the mask. Return flow tube 312 and/or outflow tube 311 may be integrally included within strap 304 or may be separate structures.

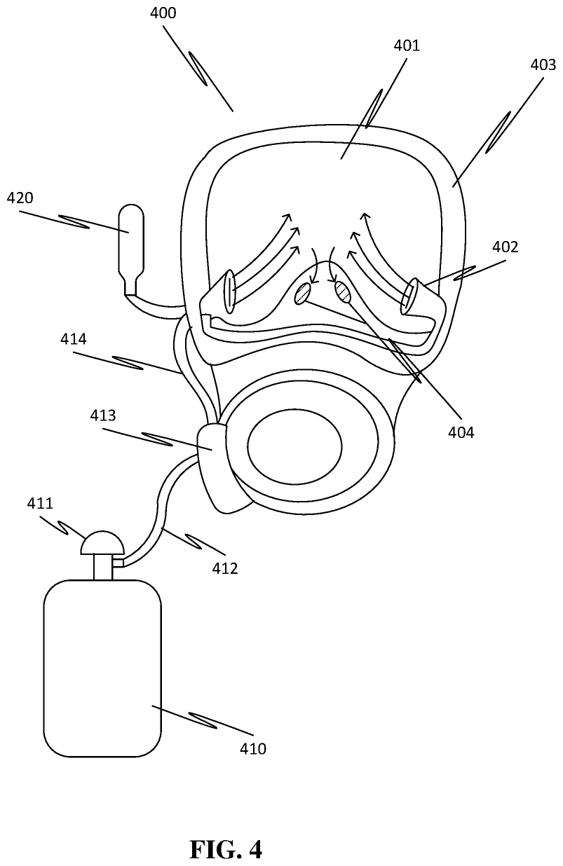

Referring now to FIG. 4, an alternative embodiment of the present disclosure is shown. FIG. 4 depicts a self-contained breathing apparatus (SCBA) 400. SCBA apparatus 400 is generally used to protect users against oxygen deficiency, dust, gases, and vapors, particularly aboard vessels, in fires, in tunnels, and in other hazardous materials (HAZMAT) environments. SCBA apparatus 400 may generally include lens 401, one or more nozzles 402, mask gasket 403, and ventilation holes 404. Nozzles 402 may be integrally attached to mask gasket 403 or may be separate pieces. Lens 401 may be fed air via nozzles 402 from one of several sources. Mask gasket 403 may include a central point for receiving air to be distributed through multiple nozzles 402. Mask gasket 403 may include a gland to allow air to pass through mask gasket 403 or another face sealing material.

Nozzles 402 may receive air from a compressed air tank 410. Compressed air tank 410 may include primary regulator 411 for adjusting a characteristic of air transported from compressed air tank 410, such characteristics including a pressure, a velocity, or a quality of the air. Air may flow via compressed air supply line 412 directly to nozzle 402 or may proceed to a secondary regulator 413. In some embodiments, the air flow may come from a specific anti-fog regulator or from a main breathing secondary regulator 413.

In exemplary embodiments, secondary regulator 413 may act as a pressure-demand regulator to keep a positive pressure in the protective area. Secondary regulator 413 may also apply additional down-line adjustments to the pressure of velocity of air transported from compressed air tank 410. Air may then flow to nozzle 402 via low pressure line 414. Low pressure line 414 may have distinct characteristics from air supply line 412; for example, it may be made of a more flexible material as the air transported therethrough may not be as pressurized as air transported through air supply 412.

In some embodiments, air may be fed through nozzles 402 by compressed air cartridge 420. Compressed air cartridge 420 may allow for spot clearing of lens 401. This embodiment may be particularly desirable in high-pressure systems.

As in the other embodiments described herein, nozzles 402 may be shaped to create air flow intended to clear or prevent condensation from forming on lens 401 and to encourage or maintain an air foil across the lens surface. Nozzles 402 may be installed analogously to the installations described in other embodiments herein.

Air may leave the protective area through any appropriate means, such as ventilation holes 404. Ventilation holes 404 may include filters or one-way air valves to prevent hazardous material from entering the protective area.

In some embodiments, SCBA 400 may further include one or more sensors, such as temperature or humidity sensors. These sensors may transmit data to a regulator to adjust the speed of air fed through nozzles 402, in manners similar to those described analogously in FIG. 1B.

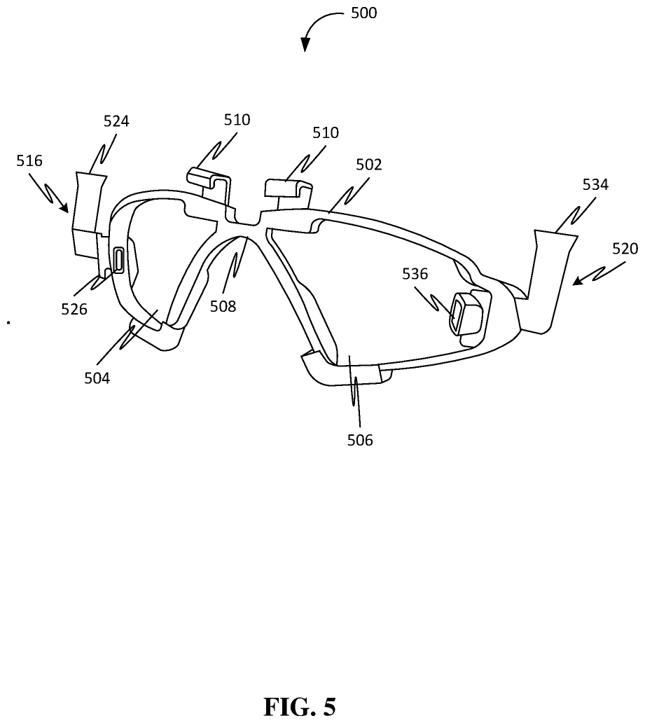

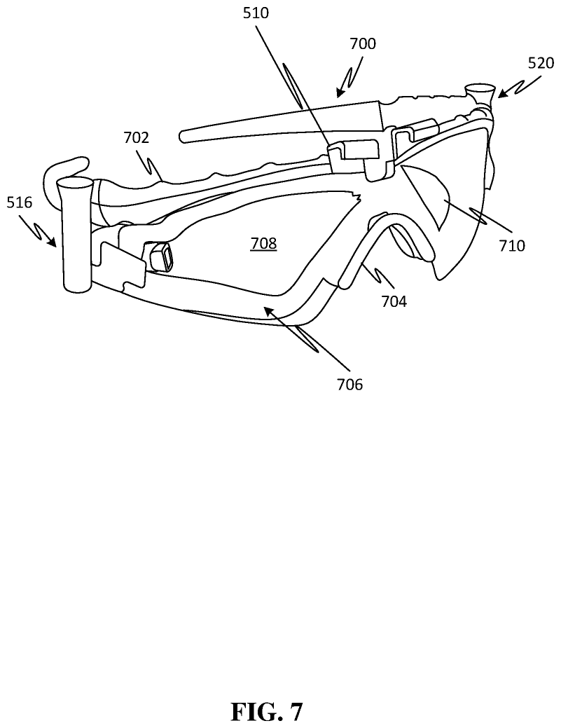

Referring now to FIG. 5, an exemplary embodiment of a modular insert for protective eyewear inserted and secured to protective eyewear and attached to an external air source is shown. An air flow Gasket 500 includes a body 502 and the air flow inserts that may be made of a soft flexible material that has sufficient rigidity to support itself against gravity without collapsing. Such a material may be 3D printed or injection molded, or otherwise manufactured with additive manufacturing techniques. A non-limiting example of the material include plastics in the family of thermoplastic elastomers (TPEs). These TPEs include thermoplastic polyurethane (TPU) compounds in various Shore Hardness ranges between Shore 80A and Shore 95A, for example. Other suitable materials for this purpose include polyethylene terephthalate glycol (PETG), polylactic acid plastics (PLA), acrylonitrile butadiene (ABS), and nylon-based materials including polyamide 11 and 12 materials. Combinations of these materials may also be employed in accordance with an embodiment of this disclosure. The body 502 may include a right eye window portion 504, a left eye window portion 506, and a nose bridge portion 508 connecting the right and left eye window portions 504, 506. A front clip 510 is attached to the body 502 at or near the nose bridge portion 508 and may be used to secure the insert to the frame 702 of the protective eyewear 700 as shown in later presented FIG. 7. This front clip 510 may have two curving legs connected to the body 502, wherein each leg is connected to the other leg by a curved connecting portion having a curve that may be counter to the curve of the curving legs. As shown in FIG. 7, the curving legs may wrap around the frame 702 and the curved connecting portion may press against the lens portion 706 of the protective eyewear 700. The lens portion 706 may include a right-side lens 708 and a left-side lens 710. In this nonlimiting embodiment, the clip 510 may have a u-shape; however, clip 510 may have other suitable shapes. In some examples, a first air flow insert 516 may be located or attached on the body 502. The first air flow insert 516 may include a first nozzle 526 and a first air flow receiver 524. A second air flow insert 520 may be located on the other side of the body 502. The second air flow insert 520 may include a second nozzle 536 and a second air flow receiver 534.

When attached to the frame 702 of the protective eyewear, the nose bridge portion 508 may rest securely on a nose bridge 704 of the protective eyewear and the clip 510 may hold the body 502 to the frame 702 so that the nose bridge portion 508 is maintained in a state pressed against the nose bridge 704 of the protective eyewear. When in this position, the right eye window portion 504 and the left eye window portion 506 are appropriately aligned with the right lens portion 708 and the left lens portion 710 of the protective eyewear 700 so vision is not obstructed by the body 502 of the air flow Gasket 500. Preferably, the body 502 of the insert is configured so it matches the configuration of the frame of the protective eyewear as shown in FIG. 7 so the body 502 and frame 702 are substantially aligned together along the top portion of the body 502. This alignment may be possible because the body 502 may have sufficient rigidity against gravity that it does not substantially sag when the clip 510 holds the body 502 to the frame 702. In a non-limiting embodiment, only the clip 510 and the nose bridge portion 508 serve to attach the air flow Gasket 500 to the protective eyewear 700.

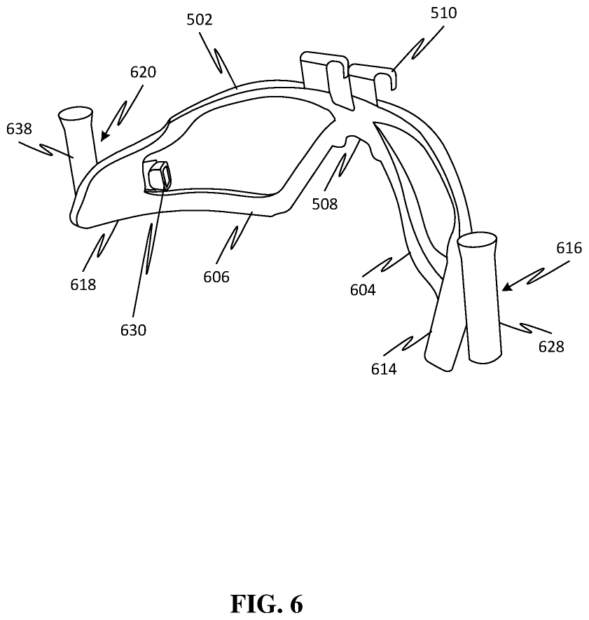

Referring to FIG. 6 a rear view of an exemplary embodiment is illustrated. Attached to a lateral corner of the right eye window portion 604 is the right-side platform portion 614 to which a first air flow interface 616 is attached. Attached to a lateral corner of the left eye window portion 606 is the left-side platform portion 618 to which a second air flow interface 620 is attached. Air flow interface 616 may be provided with an air flow conduit formed therein in order to provide a continuous passageway that runs between a first end and a second end. The first end may be formed in a receptacle 638 adapted to attach to a source of external air flow, such as to attach to a first tube attached to an external fan. The second end may be formed in a nozzle 630, wherein the nozzle is disposed to direct air flow along the inner surface of the corresponding left lens.

A second air flow interface may also be provided with an air flow conduit formed therein in order to provide a continuous passageway that runs between a first end and a second end. The first end may be formed in a receptacle 628 adapted to attach to a source of external air flow, such as to a second tube (not shown) attached to the external fan or to a second external fan. The second end may be formed in a nozzle (not shown), wherein the nozzle is disposed to direct air flow along the inner surface of the corresponding right lens.

Thus, in accordance with an embodiment of this disclosure, air flow is actively provided by an external air source, such as an air pump or fan, to the air flow interfaces 616, 620, and this air flow travels through passageways and exits nozzles respectively in order to provide an active flow of air along the lenses such as 708, 710 of FIG. 7 of the protective eyewear 700. In this way, the active air flow mitigates moisture from the vicinity of the lenses 708, 710 condensing on the lenses 708,710 while the protective eyewear is worn, which alleviates and/or prevents fogging of the lenses 708,710 in the first place. This active air flow is substantially more efficient at preventing or reversing moisture condensation on or near the lenses 708, 710 than may be achieved with passive ventilation openings. In this context, active air flow is provided by a powered device, such as a pump or fan, whereas passive air flow is provided by ventilation openings without the assistance of any powered device.

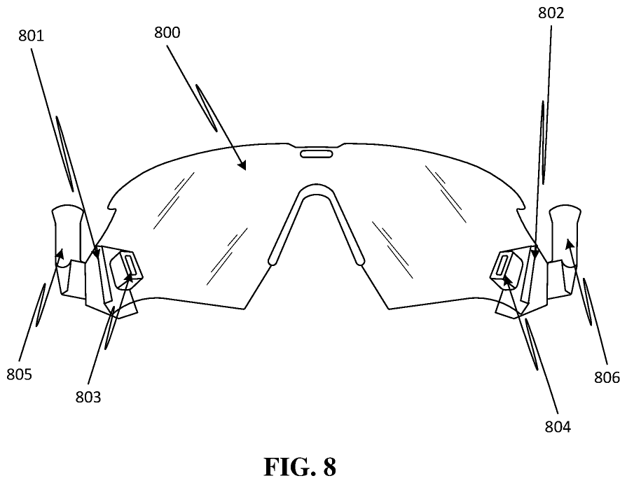

Referring now to FIG. 8, an illustration where air flow inserts used only with a protective lens and not with ancillary structure to support the air flow insert. Although the structure of an air flow insert has been discussed, to be clear an air flow insert may be include an air flow receiver (which may have been referred to previously air flow tube holder), a nozzle (as has been discussed in some detail in other examples) and components that allow these features to be attached to either a gasket, face seal, or directly to any other part of protective eyewear, such as directly to the frame or lenses. Proceeding to FIG. 8, the air flow inserts may attach directly into the lens with no supporting gasket or face seal. A lens 800 without supporting gasket or face shield may be configured to have two air flow inserts 801,802 affixed to the lens structure. The air flow inserts 801,802 may be affixed with an adhesive in some examples. Other means to affix the air flow inserts may include clips, snaps, screws (which may penetrate holes in the lens body), magnetic structures and such examples that may be able to hold the air flow inserts 801,802 to the lens 800. As in previous examples, when the air flow insert is held in place, the nozzles 803,804 may be held in specific alignment with the lens 800 surfaces so that an optimal flow of air from the nozzles 803,804 occurs across the lens 800. Corresponding air flow receivers 805 and 806 which are part of the air flow inserts 801 and 802 may provide the interface for tubes to be connected which provide air flow in the manners as has been described. In some examples, a portion of the lens body may function as a housing, and may include features that attach to straps, that support tubing pieces, that support regulators and the like. In some examples, the air flow inserts may be consider the housing or parts of the housing of the eye protection device.

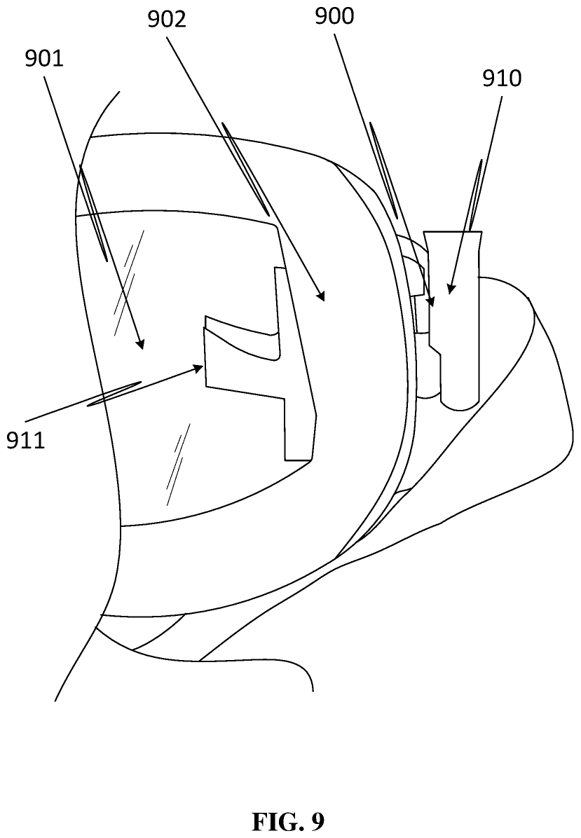

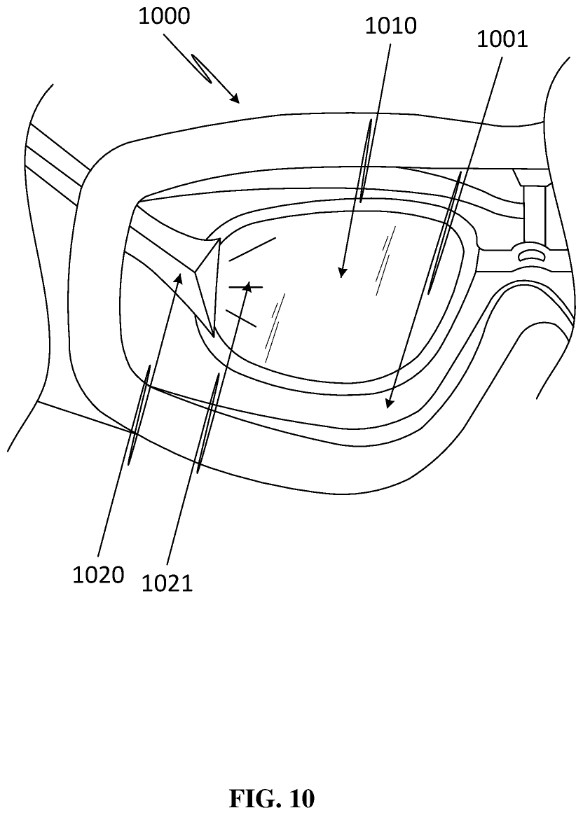

Proceeding now to FIG. 9, an illustration of air flow inserts 900 that are used in conjunction with a full goggle/mask 901, such as for example a ski goggle, with a face seal 902 is illustrated. In some examples these type of masks/goggles may have a gasket that is permanently integrated with the protective lens. In some examples, the air flow insert may be interfaced with the gasket of the protective lens. As in previous examples, the air flow insert 900 may include a portion to act as an air flow receiver 910. Furthermore, the air flow insert 900 may include a portion to act as a nozzle. The gland and gasket portion of the mask/goggle may be used to hold the nozzle 911 in a fixed position to direct air flow in an optimal manner across the face shield.

In many examples that have been discussed, it may be possible to configure a custom or prescription lens optic as part of the lens element of the various masks/shields and goggles. Referring now to FIG. 10, an illustration of a particular type of goggle 1000 is illustrated with an exemplary prescription lens optic 1010 incorporated into the lens 1001. Also illustrated is a type of nozzle 1020 that may provide air flow 1021 in optimal manners to the region of the inner surface of the prescription lens optic 1010. In some examples, the prescription lens optic 1010 may be formed into the lens 1001 itself. In other examples, the prescription lens optic 1010 may be attached with optical grade adhesives to the lens 1001. In still further examples, the prescription lens optic 1010 may be held in place by hangers within the mask body. The design of the nozzle 1020 may be altered to allow for the nozzle to be focused on the prescription lens optic 1010 as may be observed by the relatively long extension as illustrated.

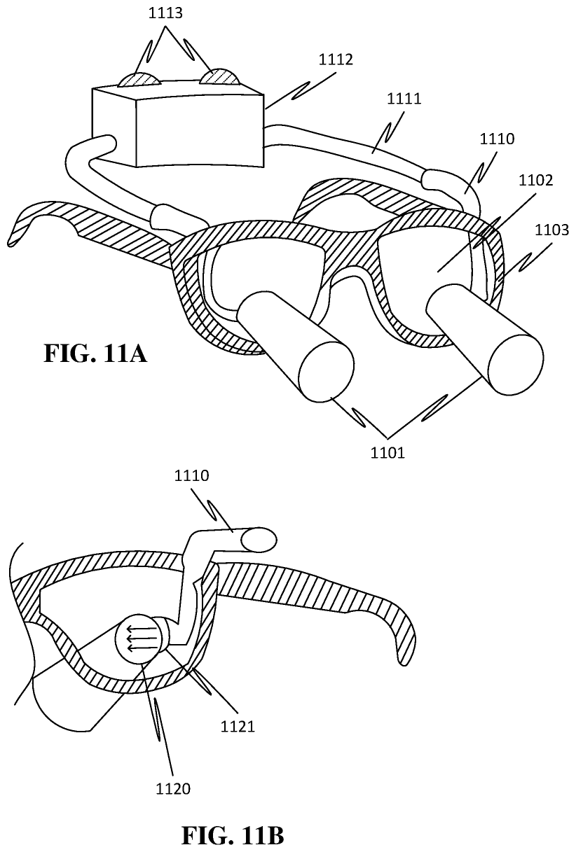

Proceeding to FIG. 11A, a specialized case of loupe optics attached to a mask or goggle base is illustrated. In some examples, the loupe optic 1101 may be attached to a lens piece 1102 held in a glass frame 1103 as a specific example. A similar configuration may be made with respect to the previously disclosed masks and goggles. In the illustration of FIG. 11A, a tube holder 1110 (which may be referred to as an air receiver) may interface with an air supply tube 1111 in fluid connection with an air flow supply 1112. The air flow supply may include the various diversity of different examples as have been discussed in relationship to air flow supplies and regulators. Various control features 1113, such as buttons, may be located upon the air flow supply 1112. As in previous examples, control of the air flow supply 1112 may also be accomplished with wired or wireless control from an external device such as a smartphone, handheld remote, or a nearby computer system. Various sensors may also be configured into this type of example and may be used to control and alter conditions of the air flow supply 1112.

Proceeding now to FIG. 11B, a closeup view from the backside of the loupe optic example is provided. The tube holder 1110 (which may also be called an air receiver) may guide air from an attached tube to the nozzle 1121. The nozzle 1121 may be focused on guiding and shaping air flow across the lens front 1120 of the loupe optic. As in previous discussions, the air flow insert including the tube holder 1110 and the nozzle 1121 may be held in a specific orientation that the air flow is directed across the lens front 1120 of the loupe optic in an optimal manner as has been described.

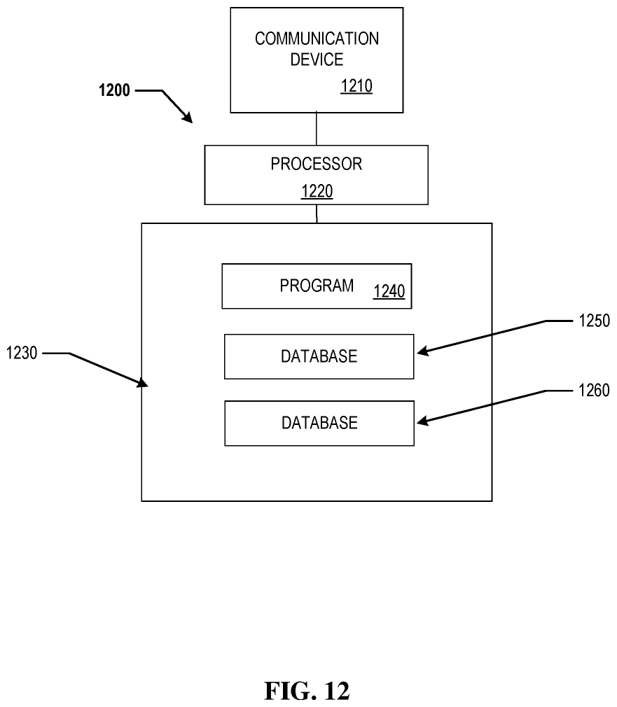

Referring now to FIG. 12, an automated controller is illustrated that may be used to implement various aspects of the present disclosure, in various embodiments, and for various aspects of the present disclosure, controller 1200 may be included in one or more of: a wireless tablet or handheld device, a server, a rack mounted processor unit. The controller may be included in one or more of the apparatus described above, such as a wireless sensor (e.g., temperature and humidity sensor) or within a regulator. The controller 1200 includes a processor unit 1220, such as one or more semiconductor based processors, coupled to a communication device 1210 configured to communicate via a communication network (not shown in FIG. 12). The communication device 1210 may be used to communicate, for example, via a distributed network such as a cellular network, an IP network, the Internet, or other distributed logic communication network.

The processor 1220 is also in communication with a storage device 1230. The storage device 1230 may include any appropriate information storage device, including combinations of digital data storage devices (e.g., solid state drives and hard disk drives), optical storage devices, and/or semiconductor memory devices such as Random Access Memory (RAM) devices and Read Only Memory (ROM) devices.

The storage device 1230 can store a software program 1240 with executable logic for controlling the processor 1220. The processor 1220 performs instructions of the software program 1240, and thereby operates in accordance with the present disclosure. The processor 1220 may also cause the communication device 1210 to transmit information, including, in some instances, control commands to operate apparatus to implement the processes described above. The storage device 1230 can additionally store related data in a database 1250 and database 1260, as needed.

### Methodology for Lens Defogging and Anti-Fogging

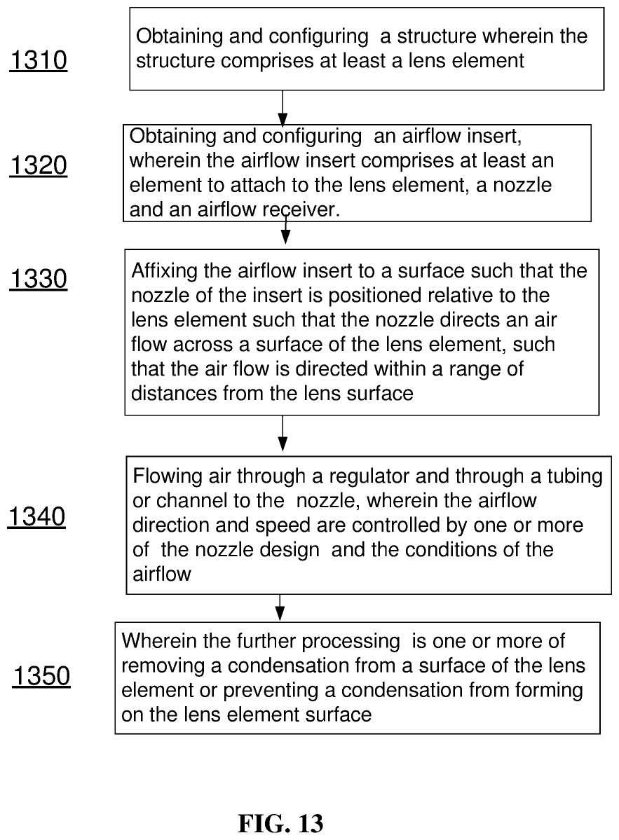

There may be numerous manners to employ and produce the various examples as have been described. Referring now to FIG. 13, an exemplary method is illustrated for the end result of either keeping a lens surface free from condensation, or removing a level of condensation from a lens surface under various operating conditions as are described in more detail following. At step 1310 in some examples a method may include obtaining a structure wherein the structure includes at least a lens element. As discussed previously there may be numerous types of structure that may apply such as in a non-limiting sense, masks, face shields, goggles, and separate eye goggles, masks for self-contained breathing apparatus and the like. The structure may define open environments or closed environments. The structure may support other ancillary equipment such as regulators, controls, tubing, straps, glands, gaskets, gas bottles and any of the components as have been described in previous sections.

In an example, at step 1320 the method may include obtaining an air flow insert, wherein the air flow insert includes at least an element to attach to the lens element, a nozzle, and an air flow receiver. As described previously, the attachment may be made to the structure with adhesives, snaps, clips, screws, and other hardware. In some cases, the structure may include just a lens piece itself to which the air flow insert may be attached. In each of these cases at least one lens and in some cases, two lenses may be equipped with lens inserts with nozzles that direct air flow at the surface of the lens. In other examples, the structure may include a nozzle that is molded or built into the structure itself.

At step 1330 the method may include affixing the air flow insert to a surface such that the nozzle of the insert is positioned relative to the lens element such that the nozzle directs an air flow across a surface of the lens element, such that the air flow is directed within a range of distances from the lens surface. As described previously, the attachment may be made to the structure with adhesives, snaps, clips, screws, and other hardware. In some cases, the structure may include just a lens piece itself to which the air flow insert may be attached. In each of these cases at least one lens and in some cases, two lenses may be equipped with lens inserts with nozzles that direct air flow at the surface of the lens. In other examples, the structure may include a nozzle that is molded or built into the structure itself.

At step 1340 the method may include flowing air through a regulator and through tubing or channels to the nozzle, wherein the air flow direction and speed are controlled by one or more of the nozzle design and the conditions of the air flow controlled by the regulator. At step 1350 the method may include further processing wherein one or more of removing a condensation from a surface of the lens element or preventing a condensation from forming on the lens element surface is performed.

In various examples of methodology, the methods may include clearing fogged surfaces or preventing them from fogging in ways and under conditions where other designs and methods fail to perform. A fogged lens may be cleared by directing air flow through a nozzle to the lens surface and clearing the lens in short times. In some examples a lens may be defogged in less than a minute or in less than two minutes. In some examples, a fogged lens where the internal surface of the fogged lens may be at a temperature different from the air resident in the structure including at least the lens element by up to a degree centigrade, or more than 1 degree centigrade, or more than 2 degrees centigrade, or more than 3 degrees centigrade or more than 5 degrees centigrade may still be cleared by flowing air through a nozzle onto the lens surface. In some examples, a difference in temperature between an inside environment of a mask and the outside environment may be 60 degrees centigrade or more. In still further examples, the range of differences in temperature between an outside environment and an internal environment may exceed 120 centigrade. Still further examples may include the lens being cleared of condensation when the relative humidity is between 50% and 75%, or between 75% and 80%, or between 80% and 85%, or between 85 and 90%, or between 90% and 95%, or greater than 95% by flowing air in a directed manner by a nozzle at the internal surface of the lens. In some examples, a combination of the various physical condition values may also be cleared by flowing air at a relatively low flow rate in a directed manner by a nozzle at the internal surface of the lens. In some examples, the air flow rate may be equivalent to replacing the air volume with the mask, goggle or goggles in less than 5 minutes, or in less than 2 minutes, or in less than 1 minute or in less than 30 seconds or in less than 10 seconds and the air flow in any particular selection of these flow regimes may still result in defogging a lens due to the directing of the air flow by the nozzle surface of the lens. In some examples, this directing of the air flow to the lens surface may result in a reduced pressure at a region near the interface of the lens surface with the air flow.

In some examples, a lens may be clear of condensation and be used by an active user. In the various methods of directing the air flow to a lens surface with the use of the nozzle in a controlled manner where the air flow rate establishes the air flow control to run along the surface of the lens and reduces the pressure in the region of the surface of the lens, the amount of time that the lens may be kept clear of condensation may be for periods of time as long at 5 minutes, or as long as 10 minutes or as long as 20 minutes or as long as 1 hour, or as long as 2 hours, or as long as 5 hours, or as long as air flow is maintained under various challenging temperature and humidity conditions including the various ranges mentioned previously.

In some examples, a lens may be logically divided into a portion that is within an optic zone and a portion that is outside of the optic zone. The concept of the optic zone may be that when a user utilizes a device including a lens and looks through the lens portion of the device, the user may not actually perceive portions of an image proceeding through the entire body of the lens. In some examples, a portion of the light rays impinging of the lens proceed through the eye of the user and are perceived as an image. That portion of the lens surface through which the perceived image passes through may be called an optic zone. In some examples, an antifogging function may occur within this optic zone. In other examples, an antifogging function may occur within a larger zone which includes the optic zone.

In some examples, a flow regime for the air flow may be established based on a given dew point of the operating condition proximate to a lens surface. Relative humidity, temperature and pressure are variables that may be optimized to control defogging in a given operating condition. Ultimately, in some examples, an important aspect may be to control the relative pressure at the lens surface through the adjustment or setting of these variables.

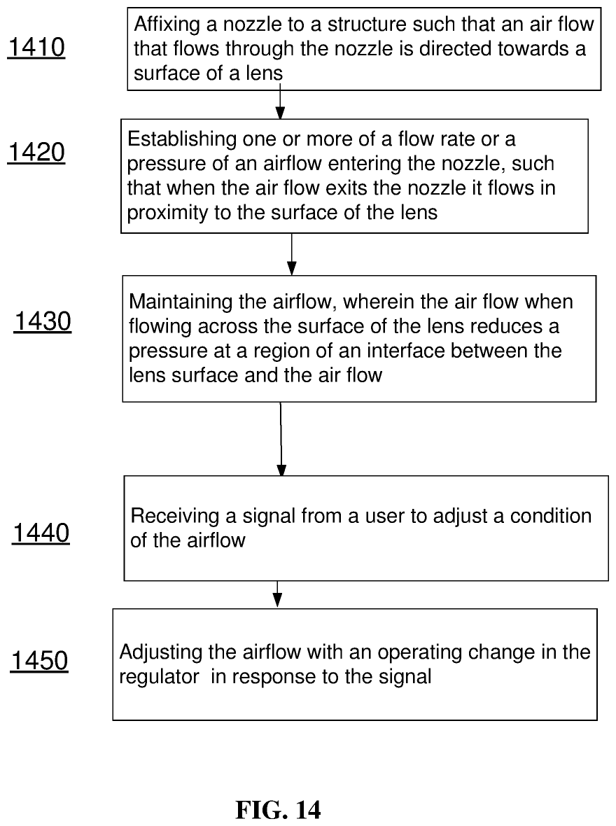

Proceeding to FIG. 14 another example method is illustrated. The various operating ranges and performance aspects as have been discussed with reference to the methodology in FIG. 13 apply for the example in FIG. 14 as well. At step 1410 in some examples a method may include affixing a nozzle to a structure such that an air flow that flows through the nozzle is directed towards a surface of a lens.

In an example, at step 1420 the method may include establishing one or more of a flow rate or a pressure of an air flow entering the nozzle, such that when the air flow exits the nozzle it flows in proximity to the surface of the lens.

At step 1430 the method may include maintaining the air flow, wherein the air flow when flowing across the surface of the lens reduces a pressure at a region of an interface between the lens surface and the air flow.

At step 1440 the method may include receiving a signal from a user to adjust a condition of the air flow.

At step 1450 the method may include adjusting the air flow with an operating change in the regulator in response to the signal.

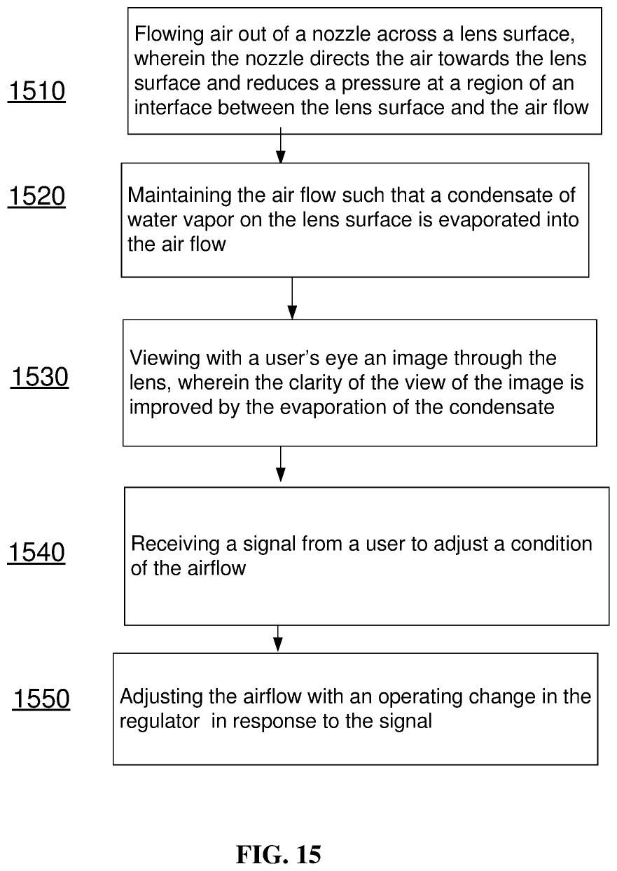

Proceeding to FIG. 15 another example method is illustrated. The various operating ranges and performance aspects as have been discussed with reference to the methodology in FIG. 13 apply for the example in FIG. 15 as well. At step 1510 in some examples a method may include flowing air out of a nozzle across a lens surface, wherein the nozzle directs the air towards the lens surface and reduces a pressure at a region of an interface between the lens surface and the air flow.

In an example, at step 1520 the method may include maintaining the air flow such that a condensate of water vapor on the lens surface is evaporated into the air flow.

At step 1530 the method may include viewing with a user's eye an image through the lens, wherein the clarity of the view of the image is improved by the evaporation of the condensate.

At step 1540 the method may include receiving a signal from a user to adjust a condition of the air flow.

At step 1550 the method may include adjusting the air flow with an operating change in the regulator in response to the signal.

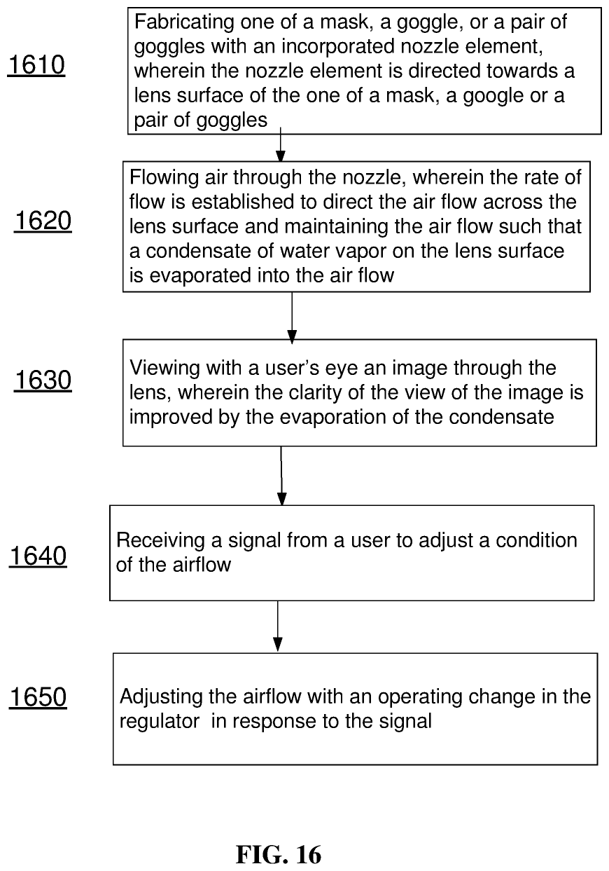

Proceeding to FIG. 16 another example method is illustrated. The various operating ranges and performance aspects as have been discussed with reference to the methodology in FIG. 13 apply for the example in FIG. 16 as well. At step 1610 in some examples a method may include fabricating one of a mask, a goggle, or a pair of goggles with an incorporated nozzle element, wherein the nozzle element is directed towards a lens surface of the one of a mask, a google or a pair of goggles.

In an example, at step 1620 the method may include flowing air through the nozzle, wherein the rate of flow is established to direct the air flow across the lens surface and maintaining the air flow such that a condensate of water vapor on the lens surface is evaporated into the air flow.

At step 1630 the method may include viewing with a user's eye an image through the lens, wherein the clarity of the view of the image is improved by the evaporation of the condensate.

At step 1640 the method may include receiving a signal from a user to adjust a condition of the air flow.

At step 1650 the method may include adjusting the air flow with an operating change in the regulator in response to the signal.

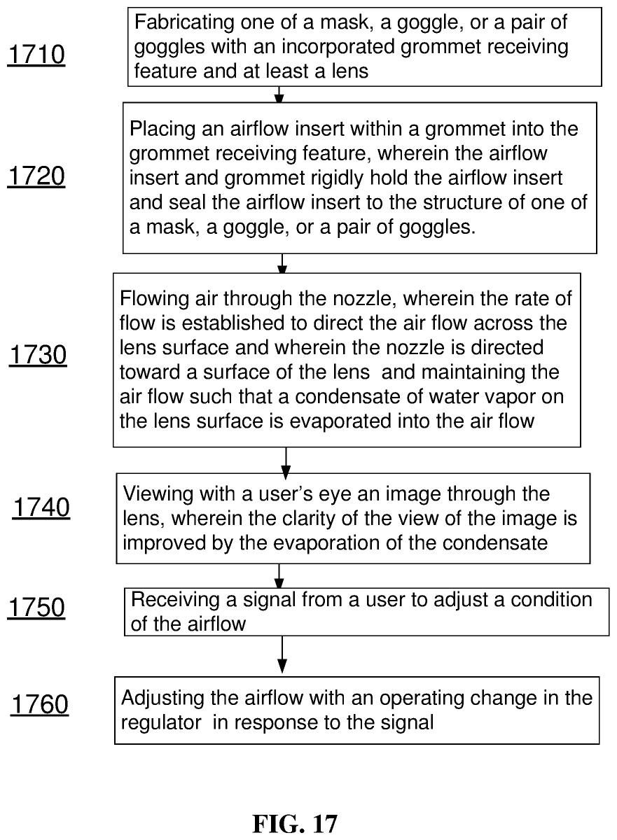

Proceeding to FIG. 17 another example method is illustrated. The various operating ranges and performance aspects as have been discussed with reference to the methodology in FIG. 13 apply for the example in FIG. 17 as well. At step 1710 in some examples a method may include fabricating one of a mask, a goggle, or a pair of goggles with an incorporated grommet receiving feature and at least a lens.

In an example, at step 1720 the method may include placing an air flow insert within a grommet into the grommet receiving feature, wherein the air flow insert, and grommet rigidly hold the air flow insert and seal the air flow insert to the structure of one of a mask, a goggle, or a pair of goggles.

At step 1730 the method may include flowing air through the nozzle, wherein the rate of flow is established to direct the air flow across the lens surface and wherein the nozzle is directed toward a surface of the lens and maintaining the air flow such that a condensate of water vapor on the lens surface is evaporated into the air flow.

At step 1740 the method may include viewing with a user's eye an image through the lens, wherein the clarity of the view of the image is improved by the evaporation of the condensate.

At step 1750 the method may include receiving a signal from a user to adjust a condition of the air flow.

At step 1760 the method may include adjusting the air flow with an operating change in the regulator in response to the signal.

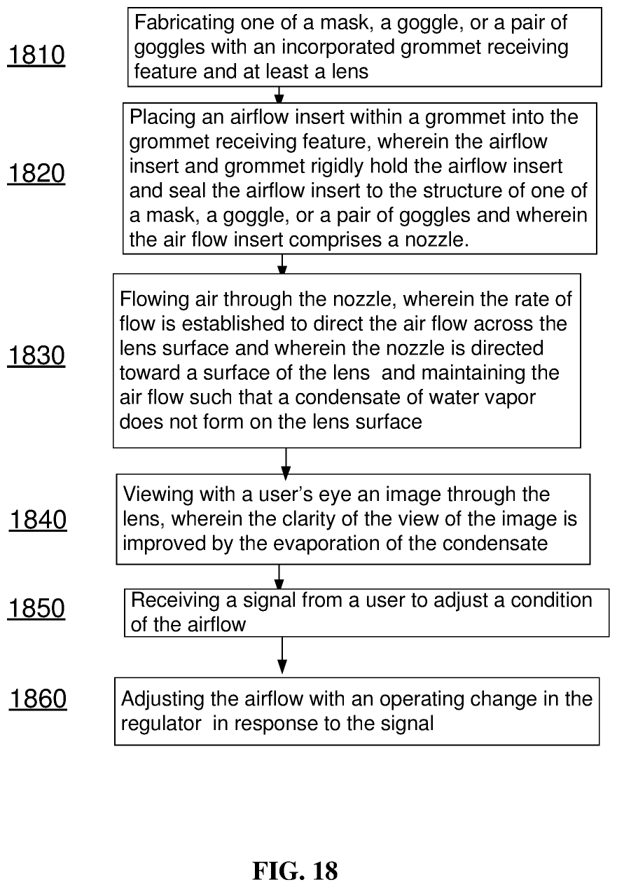

Proceeding to FIG. 18 another example method is illustrated. The various operating ranges and performance aspects as have been discussed with reference to the methodology in FIG. 13 apply for the example in FIG. 18 as well. At step 1810 in some examples a method may include fabricating one of a mask, a goggle, or a pair of goggles with an incorporated grommet receiving feature and at least a lens.

In an example, at step 1820 the method may include placing an air flow insert within a grommet into the grommet receiving feature, wherein the air flow insert, and grommet rigidly hold the air flow insert and seal the air flow insert to the structure of one of a mask, a goggle, or a pair of goggles and wherein the air flow insert includes a nozzle.

At step 1830 the method may include flowing air through the nozzle, wherein the rate of flow is established to direct the air flow across the lens surface and wherein the nozzle is directed toward a surface of the lens and maintaining the air flow such that a condensate of water vapor does not form on the lens surface.

At step 1840 the method may include viewing with a user's eye an image through the lens, wherein the clarity of the view of the image is improved by the evaporation of the condensate.

At step 1850 the method may include receiving a signal from a user to adjust a condition of the air flow.

At step 1860 the method may include adjusting the air flow with an operating change in the regulator in response to the signal.

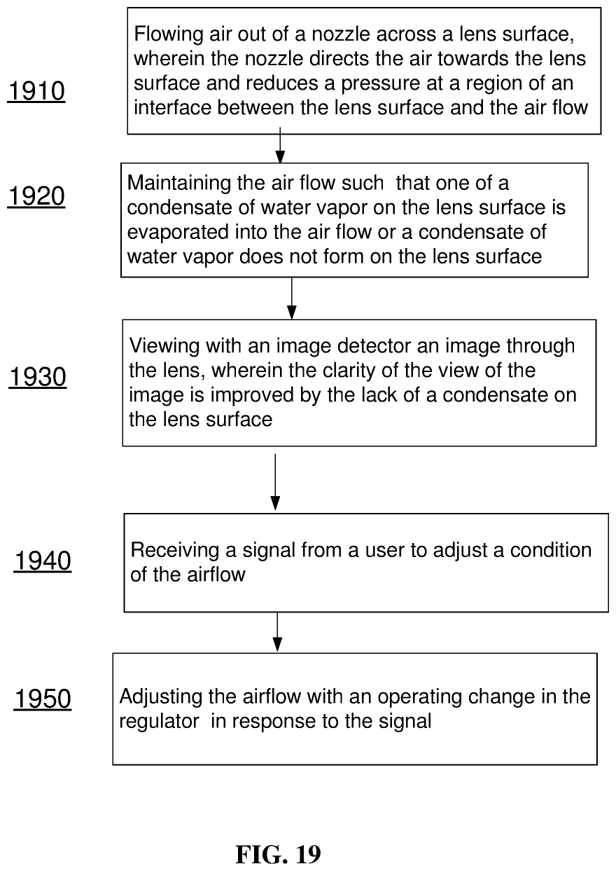

Proceeding to FIG. 19 another example method is illustrated. The various operating ranges and performance aspects as have been discussed with reference to the methodology in FIG. 13 apply for the example in FIG. 19 as well. At step 1910 in some examples a method may include flowing air out of a nozzle across a lens surface, wherein the nozzle directs the air towards the lens surface and reduces a pressure at a region of an interface between the lens surface and the air flow.

In an example, at step 1920 the method may include maintaining the air flow such that one of a condensate of water vapor on the lens surface is evaporated into the air flow or a condensate of water vapor does not form on the lens surface.

At step 1930 the method may include viewing with an image detector eye an image through the lens, wherein the clarity of the view of the image is improved by the lack of a condensate on the lens surface.

At step 1940 the method may include receiving a signal from a user to adjust a condition of the air flow.

At step 1950 the method may include adjusting the air flow with an operating change in the regulator in response to the signal.

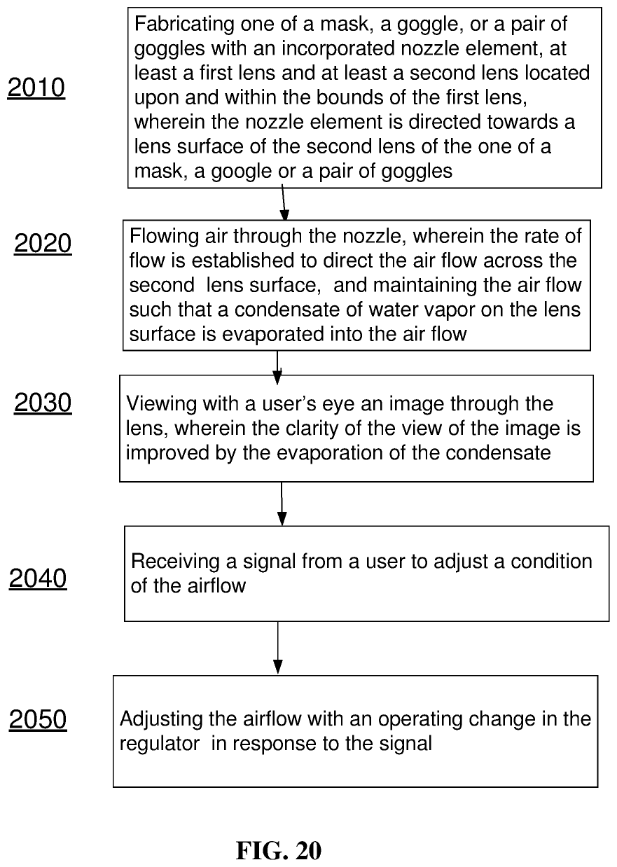

Proceeding to FIG. 20 another example method is illustrated. The various operating ranges and performance aspects as have been discussed with reference to the methodology in FIG. 13 apply for the example in FIG. 20 as well. At step 2010 in some examples a method may include fabricating one of a mask, a goggle, or a pair of goggles with an incorporated nozzle element, at least a first lens and at least a second lens located upon and within the bounds of the first lens, wherein the nozzle element is directed towards a lens surface of the second lens of the one of a mask, a google or a pair of goggles.

In an example, at step 2020 the method may include flowing air through the nozzle, wherein the rate of flow is established to direct the air flow across the second lens surface, and maintaining the air flow such that a condensate of water vapor on the lens surface is evaporated into the air flow.

At step 2030 the method may include viewing with a user's eye an image through the lens, wherein the clarity of the view of the image is improved by the evaporation of the condensate.

At step 2040 the method may include receiving a signal from a user to adjust a condition of the air flow.

At step 2050 the method may include adjusting the air flow with an operating change in the regulator in response to the signal.

### Glossary of Selected Terms

Air Driver—As used herein an Air Driver is an apparatus that effectuates or causes air flow in the system. The air driver's rate of air flow may commonly be controlled by a Regulator. The Air Driver may cause the movement of air and may include but not be limited to, an electric impeller, blower fan, an axial fan, and a piezoelectric fan. The air driver may also be a compressed source of gas (e.g., a tank of air) where a Regulator-controlled air valve governs the rate of the gas flow.

Air flow Insert As used herein an air flow insert is an apparatus that will insert into or otherwise attach to a gasket or a face seal that provides specialized air flow onto the inside and/or outside of the protective eyewear. An Air flow Insert includes an air flow receiver (aka air flow tube holder), a nozzle and components that allow it to be attached to either a gasket, face seal, or directly to any other part of protective eyewear, such as directly to the frame or lenses, that allow the nozzle to provide air flow in a particular direction and velocity such that it prevents condensation and therefore increases visual acuity for the user. An air flow insert may also be used to channel air flowing out of the eye protection device. When used, the output” air flow insert may be attached to an air flow return line.

Eye Protection Device—As used herein an eye protection device is an apparatus that is used to provide protection of a user's eye while providing an ability to view through portions of the apparatus in front of the eye. In some examples, an eye protection device may include a mask with a mask housing with one or more lens, a face seal or gasket with glands to which are attached one or more nozzles. The face seal (or gasket) may provide an enclosed space between the user's face and the mask and its mask housing.

Face seal—As used herein a face seal is a part of a protective facial apparatus that is generally permanently integrated with the protective lens or must be in place for the protective facial apparatus to operate effectively in normal operations. If a face seal is not permanently integrated with the mask housing, it may also be termed a Gasket.

Gasket As used herein a gasket is an attachable/removeable component that, when attached to protective eyewear, creates a protective face seal between the user's face and the eye protection device.

Gland—As used herein a gland is a component or apparatus that is used when a cable or tube (e.g., an air flow supply tube) needs to pass through some bulkhead (e.g., a panel, housing or, in our context, a gasket or face seal) while still preserving an operational integrity of the bulkhead. For example, an input air flow gland, in the context of the invention, may be a mechanism by which air flow supply can penetrate through a protective gasket or face seal via an air flow Insert in such a way as to not compromise the watertight, airtight, or other operational sealing requirement of said gasket or face seal. Similarly, an output air flow gland may provide a means to locate an air flow insert through which air exits the protective eyewear.

Mask Housing—As used herein a mask housing is a frame that secures the lens to the mask.

Nozzle—As used herein a Nozzle is a component which creates air flow conditions to define one or more of the direction and the velocity of the air flow across the protective lenses to mitigate condensation. In some examples, a nozzle may be associated with any of the other system components (gasket, lens, frame, face seal, etc.) as long as it satisfies the parameters for the principles of operation of the nozzle as disclosed herein.

Optic Zone—As used herein an optic zone is the portion of a lens through which a user may perceive an image when the lens is positioned in a position of use.

Prescription lens frame hanger—As used herein prescription lens frame hangers are entities to hold prescription lenses and relate to people with correctable vision who may optionally mount prescription lenses in a mask, goggle or other protective eyewear using a prescription lens frame hanger.

Protective lens—As used herein the protective lens is an element formed of a material that allows light to pass to the user with great optical clarity while protecting the user from various hazards (e.g., flying objects/debris, smoke, chemicals, water, biological moieties, etc.).

Regulator—As used herein a regulator is a component or element that controls a flow of air. In some examples, a regulator may be controlled manually. In other examples, a level of automatic control may also be included in a regulator.

### CONCLUSION

A number of embodiments of the present disclosure have been described. While this specification contains many specific implementation details, there should not be construed as limitations on the scope of any disclosures or of what may be claimed, but rather as descriptions of features specific to particular embodiments of the present disclosure. While embodiments of the present disclosure are described herein by way of example using several illustrative drawings, those skilled in the art may recognize the present disclosure is not limited to the embodiments or drawings described. It should be understood the drawings and the detailed description thereto are not intended to limit the present disclosure to the form disclosed, but to the contrary, the present disclosure is to cover all modification, equivalents and alternatives falling within the spirit and scope of embodiments of the present disclosure as defined by the appended claims.

Any headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include,” “including,” and “includes” mean including but not limited to. To facilitate understanding, like reference numerals have been used, where possible, to designate like elements common to the figures.

The phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted the terms “comprising,” “including,” and “having” can be used interchangeably.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases, be excised from the combination, and the claimed combination may be directed to a sub-combination or variation of a sub-combination.

Similarly, while method steps may be depicted in the drawings in a particular order, this should not be understood as requiring that such operations be performed in the particular order shown or in a sequential order, or that all illustrated operations be performed, to achieve desirable results.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. Nevertheless, it may be understood that various modifications may be made without departing from the spirit and scope of the claimed disclosure.

What is claimed is:

1. A method of controlling a fogging condition on a lens surface, the method comprising:
flowing air out of a nozzle across the lens surface, wherein the nozzle directs the air towards the lens surface and reduces a pressure at a region of an interface between the lens surface and the flowing air; and
maintaining the flowing of air such that a condensate of water vapor on the lens surface is evaporated into the flowing air.

2. The method of claim 1 further comprising viewing with a user's eye an image through an optic zone of the lens surface, wherein a clarity of the view of the image is improved by the evaporation of the condensate.

3. The method of claim 2 wherein the lens surface is comprised within a closed environment of a mask, and wherein a rate of flowing air is less than an amount equal to a volume of the closed environment per minute.

4. The method of claim 2 wherein the lens surface is comprised within an open environment, and wherein a rate of flowing air flows the air across the surface at a rate of approximately 1.4 m/sec.

5. The method of claim 2 wherein the nozzle is comprised within an air flow insert, wherein the air flow insert further comprises an air flow receiver wherein air is provided to flow to the nozzle, and wherein the air flow insert is affixed to the lens surface.

6. The method of claim 2 wherein the nozzle is comprised within an air flow insert, wherein the air flow insert further comprises an air flow receiver wherein air is provided to flow to the nozzle, wherein the air flow insert is comprised within a mask and wherein the air flow insert passes through a bulkhead of the mask.

7. The method of claim 2 wherein the nozzle is comprised within an air flow insert, wherein the air flow insert further comprises an air flow receiver wherein air is provided to flow to the nozzle, wherein the air flow insert is comprised within a goggle and wherein the air flow insert passes through a bulkhead of the goggle.

8. A method of producing an anti-fogging apparatus, the method comprising:
configuring a structure wherein the structure comprises at least a lens element;
configuring an air flow insert, wherein the air flow insert comprises at least an element to attach to the lens element, a nozzle, and an air flow receiver; and
attaching the air flow insert to the structure; and
wherein after the attaching, the nozzle is positioned to guide air flow through the nozzle towards a surface of the lens element and across a span of an optic zone of the lens element.

9. The method of claim 8 further comprising:
attaching a regulator to the structure, wherein the regulator controls a rate of the air flow through the nozzle.

10. The method of claim 9 wherein a user control of the regulator adjusts the rate of air flow through the nozzle.

11. The method of claim 10 wherein the adjusted rate of air flow through the nozzle projects air flow in a region of surface of the lens element, and the projecting air flow reduces a pressure of air in a region proximate to the lens surface.

12. The method of claim 11 wherein a fogging of the lens surface is removed upon flowing of the air flow.

13. The method of claim 11 wherein the flowing of the air flow on the lens surface prevents a fogging of the lens surface during a use of the anti-fogging apparatus for at least an hour of usage.

14. An eye protection apparatus with a lens surface anti-fogging means, the eye protection apparatus comprising:
an eye protection housing;
a lens configured on a first portion of the eye protection housing;
a nozzle positioned proximate to the lens, the nozzle comprising a receiving portion and an outflow portion, wherein the outflow portion is positioned at a flow angle relative to the lens surface;
an air flow regulator comprising an air driver; and
an air supply line in fluid connection between the nozzle and the air flow regulator, wherein the air supply line receives a flow of air driven by the air driver and connects the flow of air to the receiving portion of the nozzle; and
wherein the flow of air proceeds through the nozzle and projects upon the lens surface; and
wherein the air flow reduces a pressure of air in a region proximate to the lens surface.

15. The eye protection apparatus of claim 14 wherein the reduction of the pressure of the air in the region proximate to the lens surface facilitates an evaporation of a condensate upon the lens surface and a reduction of a fogging.

16. The eye protection apparatus of claim 14 wherein the reduction of the pressure of the air in the region proximate to the lens surface inhibits a condensation of a condensate upon the lens surface and an inhibition of a fogging.

17. The eye protection apparatus of claim 16 further comprising a strap, wherein the strap holds the eye protection apparatus into a position configured upon a head of a user such that the user may view an image through an optic zone of the lens surface, wherein the optic zone of the optic zone of the lens surface is free of the fogging.

18. The eye protection apparatus of claim 16 wherein the lens surface is comprised within a closed environment.

19. The eye protection apparatus of claim 16 wherein the lens surface is comprised within an open environment.

20. The eye protection apparatus of claim 16 wherein the reduction of pressure of the air in the region of proximate to the lens surface is more than 5 Torr above that necessary to prevent condensation, and wherein the reduction of pressure of the air occurs across all of the optic zone of the lens surface.
